(19) United States
(12) Patent Application Publication
(10) Pub. No.: US 2025/0170382 A1
(43) Pub. Date: May 29, 2025

(54) EXTENDABLE OVERTUBE FOR A FLEXIBLE ELONGATE DEVICE

(71) Applicant: INTUITIVE SURGICAL OPERATIONS, INC., Sunnyvale, CA (US)

(72) Inventors: David Bailey, Portola Valley, CA (US); Jinsong Huang, San Jose, CA (US)

(21) Appl. No.: 18/961,760

(22) Filed: Nov. 27, 2024

Related U.S. Application Data
(60) Provisional application No. 63/603,355, filed on Nov. 28, 2023.

(51) Int. Cl. A61M 39/10

(52) U.S. Cl. CPC A61M 39/10

(57) ABSTRACT

An apparatus for movably coupling a robotic system to an airway management device includes an overtube and a connection mechanism. The overtube includes a flexible tubular portion. The connection mechanism includes a first portion configured to be receive the overtube, a second portion configured to be connected with a proximal end of the airway management device, a lumen defined within the first portion and second portion, and a locking mechanism configured to connect to the flexible tubular portion of the overtube. When the overtube is connected with the connection mechanism, the flexible tubular portion of the overtube is within the lumen of the connection mechanism and extends distal to a distal end of the airway management device. The flexible tubular portion of the overtube structurally supports a flexible elongate device when the flexible elongate device is received within flexible tubular portion.

### CROSS-REFERENCE TO RELATED APPLICATION

This application claims the priority benefit of U.S. Provisional Application No. 63/603,355, filed Nov. 28, 2023, which is hereby incorporated by reference herein in its entirety.

### FIELD

The present disclosure is directed to an airway management device providing structural support for a flexible elongate device.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments (including surgical, diagnostic, therapeutic, or biopsy instruments) to reach a target tissue location. One such minimally invasive technique is to use a flexible elongate device such as a flexible catheter, bronchoscope, or endoscope, that can be inserted into anatomic passageways and navigated towards a region of interest within the patient anatomy. The insertion of a flexible elongate device into anatomy often involves use of devices that structurally support and guide the flexible elongate device into the anatomy.

### SUMMARY

Consistent with some examples, an apparatus for movably coupling a robotic system to an airway management device includes an overtube and a connection mechanism. The overtube includes a flexible tubular portion. The connection mechanism includes a first portion, a second portion, a lumen, and a locking mechanism. The first portion is configured to receive the overtube. The second portion is configured to be connected with a proximal end of the airway management device. The lumen is defined within the first portion and second portion. The locking mechanism is configured to connect to the flexible tubular portion of the overtube. When the overtube is connected with the connection mechanism, the flexible tubular portion of the overtube is within the lumen of the connection mechanism and extends distal to a distal end of the airway management device.

In some examples, the apparatus may further include a first seal, and the first seal may be configured to seal a lumen of the flexible tubular portion of the overtube.

In some examples, the first seal may include a cross-slit valve and a septum.

In some examples, the apparatus may further include a cap for the overtube, and the cap may include the first seal.

In some examples, the apparatus may further include a second seal, and the second seal may be configured to seal a lumen of the connection mechanism.

In some examples, the second seal may include a septum seal and a cross-slit valve.

In some examples, the locking mechanism may be configured to secure the second seal around the flexible tubular portion to prevent movement of the overtube.

In some examples, the locking mechanism may include a first lock part and a second lock part. The first lock part may include a threaded base. The second lock part may include a threaded wall configured to engage the threaded base of the first lock part. The second lock part may further include a foot extending inwardly from the threaded wall. The second lock part may be positioned adjacent to the second seal. Relative movement between the threaded base of the first lock part and the threaded wall of the second lock part may cause the foot of the second lock part to compress a second seal, thereby causing the second seal to expand radially inward to engage the flexible tubular portion of the overtube.

In some examples, the locking mechanism may further include an outer housing, and the second lock part may include splines on an outside of the threaded wall. The splines may be configured to engage the outer housing so that the second lock part cannot rotate relative to the outer housing.

In some examples, the connection mechanism may be configured to be at least partially disconnected from the robotic system when motion between the robotic system and the airway management device includes an above-threshold displacement of the airway management device relative to the robotic system.

In some examples, the above-threshold displacement may create a force that overcomes a magnetic force securing the connection mechanism to the robotic system.

In some examples, the flexible tubular portion of the overtube includes an atraumatic tip.

In some examples, the flexible tubular portion has an interior surface comprising a low friction interface.

Consistent with some examples, a medical system includes a robotic system, an airway management device, and an overtube. The robotic system is configured to drive a flexible elongate device. The airway management device includes a cylindrical passageway and an engagement portion. The cylindrical passageway is configured to couple with the robotic system. The engagement portion is attached to the cylindrical passageway. The engagement portion is configured to engage a patient anatomy proximal to the trachea. The overtube includes a flexible tubular portion configured to be inserted within the cylindrical passageway of the airway management device. The flexible tubular portion is configured to extend distal to the engagement portion of the airway management device when inserted within the cylindrical passageway of the airway management device. The flexible tubular portion structurally supports a flexible elongate device when the flexible elongate device is received within flexible tubular portion.

In some examples, the robotic system may include an instrument carriage and a flexible elongate device guide configured to provide lateral support to the flexible elongate device during movement of the instrument carriage.

In some examples, the flexible elongate device guide may be configured to accommodate the overtube.

In some examples, the medical system may further include a connection mechanism. The connection mechanism may include a first portion, a second portion, a lumen, and a locking mechanism. The first portion may be configured to receive the overtube. The second portion may be configured to be connected with a proximal end of the airway management device. The lumen may be defined within the first portion and second portion. The locking mechanism may be configured to connect to the flexible tubular portion of the overtube.

Consistent with some examples, a method of positioning a flexible elongate device includes providing a connection mechanism having a first portion and a second portion. The method further includes connecting the second portion of the connection mechanism to an airway management device. The method further includes inserting, using the robotic system, a distal portion of the flexible elongate device into an anatomy a first distance such that the flexible elongate device extends through the connection mechanism and extends distal to the airway management device. The method further includes extending an overtube over the flexible elongate device such that a flexible tubular portion of the overtube extends through the connection mechanism and extends distal to the airway management device. The method further includes further inserting, using the robotic system, the flexible elongate device into the anatomy beyond the first distance, wherein the flexible tubular portion of the overtube provides structural support for the flexible elongate device distal to the airway management device.

In some examples, the method may include locking the flexible tubular portion of the overtube to the connection mechanism.

In some examples, locking the flexible tubular portion of the overtube to the connection mechanism may include sealing a lumen of the connection mechanism with a second seal

In some examples, the connection mechanism may include a locking mechanism, the locking mechanism including a first lock part including a threaded base, a second lock part including a threaded wall configured to engage the threaded base of the first lock part, and a foot extending inwardly from the threaded wall. The method may further include causing relative movement between the threaded base of the first lock part and the threaded wall of the second lock part to cause the foot of the second lock part to compress the seal, thereby causing the seal to expand radially inward to engage the flexible tubular portion of the overtube.

In some examples, the robotic system may include an instrument carriage and a flexible elongate device guide configured to provide lateral support to the flexible elongate device during movement of the instrument carriage. The method may include extending the flexible elongate device guide when the flexible elongate device is inserted into the anatomy the first distance.

In some examples, the robotic system may include an instrument carriage and a flexible elongate device guide configured to provide lateral support to the flexible elongate device during movement of the instrument carriage. The method may include retracting the flexible elongate device guide to enable insertion of the overtube over the flexible elongate device and reextending the flexible elongate device after insertion of the overtube.

In some examples, extending the overtube over the flexible elongate device may not result in extending the flexible tubular portion of the overtube distal to the flexible elongate device.

### BRIEF DESCRIPTIONS OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIG. 1 is a simplified diagram of a robotic and/or teleoperated medical system according to some embodiments.

FIG. 2A is a simplified diagram of a medical instrument system according to some embodiments.

FIG. 2B is a simplified diagram of a medical instrument with an extended medical tool according to some embodiments.

FIGS. 3A and 3B are simplified diagrams of side views of a patient coordinate space including a medical instrument mounted on an insertion assembly according to some embodiments.

FIG. 4 illustrates an example of an instrument manipulator including a flexible elongate device according to some embodiments.

FIG. 5 illustrates an airway management device according to some embodiments.

FIG. 6A illustrates an exploded view of a connection mechanism according to some embodiments.

FIG. 6B illustrates a perspective view of the connection mechanism of FIG. 6A.

FIG. 7 is a perspective view of an apparatus for movably coupling a robotic system to an airway management device according to some embodiments.

FIG. 8 is a perspective view of an overtube of the apparatus of FIG. 7 according to some embodiments including a cutaway to show an interior of the overtube.

FIG. 9 is a cross-sectional perspective view of a first seal of the apparatus of FIG. 7 according to some embodiments.

FIG. 10 is a cross-sectional view of the apparatus of FIG. 7.

FIG. 11 is a perspective view of a first lock part of a connection mechanism of the apparatus of FIGS. 7 and 10 according to some embodiments.

FIG. 12 is a perspective view of a second lock part of the connection mechanism of the apparatus of FIGS. 7 and 10 according to some embodiments.

FIG. 13 is a perspective view of a first portion of an outer housing of the connection mechanism of the apparatus of FIGS. 7 and 10 according to some embodiments.

FIG. 14 is a perspective view of a second portion of an outer housing of the connection mechanism of the apparatus of FIGS. 7 and 10 according to some embodiments.

FIG. 15 is a perspective view of a second seal of the apparatus of FIGS. 7 and 10 according to some embodiments.

FIG. 16 illustrates schematically a method of positioning a flexible elongate device using the apparatus of FIGS. 7 and 10 according to some embodiments.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

In the following detailed description of the aspects of the invention, numerous specific details are set forth in order to provide a thorough understanding of the disclosed embodiments. However, it will be obvious to one skilled in the art that the embodiments of this disclosure may be practiced without these specific details. In other instances, well known methods, procedures, components, and circuits have not been described in detail so as not to unnecessarily obscure aspects of the embodiments of the invention. And, to avoid needless descriptive repetition, one or more components or actions described in accordance with one illustrative embodiment can be used or omitted as applicable from other illustrative embodiments. For the sake of brevity, the numerous iterations of these combinations will not be described separately. For simplicity, in some instances the same reference numbers are used throughout the drawings to refer to the same or like parts.

In one example, an endotracheal tube (ET tube) may be inserted through the nose or mouth of a patient and placed within the trachea. A medical instrument or device may then be inserted through the endotracheal tube and used to view the trachea and other bronchial passages and/or to conduct a biopsy and/or diagnose lung diseases and infections. The ET tube is used for airway management, for example for use during mechanical ventilation as well for prevention of damage to patient anatomy such as vocal cords during the medical procedure. Because the ET tube extends distally into the trachea beyond the vocal cords, the ET tube provides structural support for the insertion of, for example, a flexible elongate device, providing the stability necessary for the flexible elongate device to be navigated to its target destination.

A laryngeal mask airway (LMA) may be used in place of an ET tube. An LMA includes a cuff that forms an airtight seal proximal to the vocal cords. Because the LMA does not extend into the trachea, the LMA cannot provide as much structural support for the insertion of, for example, a flexible elongate device into the lungs. Without this support, the flexible elongate device can prolapse and be unable to reach a target destination. Further, when used with certain systems, the (e.g., lateral) movement of the flexible elongate device without support distal to the vocal cords can trigger a false positive detection of patient motion by the system, causing error messages or other undesirable delays. The apparatus and methods described below address these issues to facilitate use of an LMA.

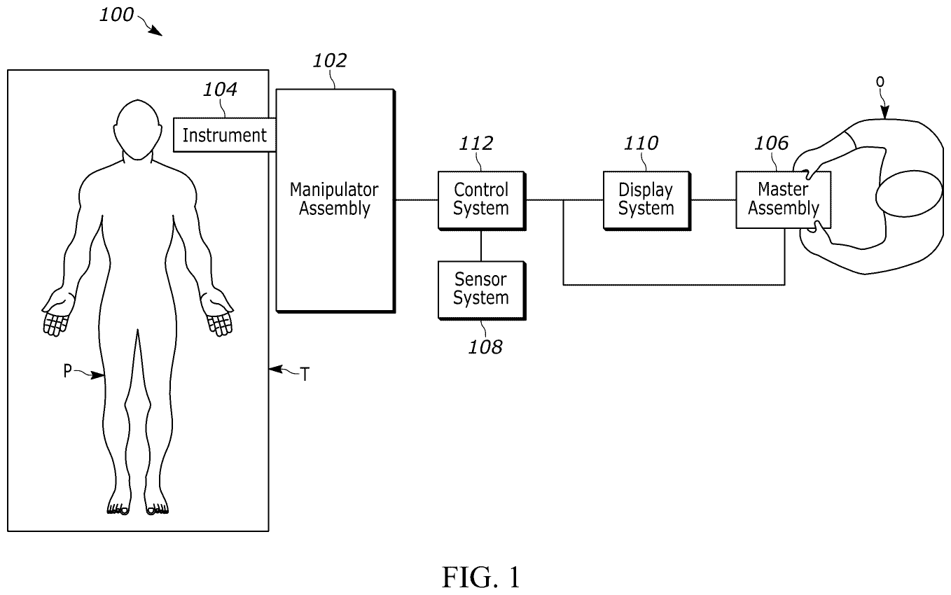

As shown in FIG. 1, medical system 100 generally includes a manipulator assembly 102 for operating a medical instrument 104 in performing various procedures on a patient P. The manipulator assembly 102 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. Manipulator assembly 102 is mounted to or near an operating table T. A master assembly 106 allows an operator O (e.g., a surgeon, a clinician, and/or a physician as illustrated in FIG. 1) to view the interventional site and to control manipulator assembly 102.

Master assembly 106 may be located at an operator console which is usually located in the same room as operating table T, such as at the side of a surgical table on which patient P is located. However, it should be understood that operator O can be located in a different room or a completely different building from patient P. Master assembly 106 generally includes one or more control devices for controlling manipulator assembly 102. The control devices may include any number of a variety of input devices, such as joysticks, trackballs, data gloves, trigger-guns, hand-operated controllers, voice recognition devices, body motion or presence sensors, and/or the like. To provide operator O a strong sense of directly controlling instruments 104 the control devices may be provided with the same degrees of freedom as the associated medical instrument 104. In this manner, the control devices provide operator O with telepresence or the perception that the control devices are integral with medical instruments 104.

In some embodiments, the control devices may have more or fewer degrees of freedom than the associated medical instrument 104 and still provide operator O with telepresence. In some embodiments, the control devices may optionally be manual input devices which move with six degrees of freedom, and which may also include an actuatable handle for actuating instruments (for example, for closing grasping jaws, applying an electrical potential to an electrode, delivering a medicinal treatment, and/or the like).

The manipulator assembly 102 supports medical instrument 104 and may include a kinematic structure of one or more non-servo controlled links (e.g., one or more links that may be manually positioned and locked in place, generally referred to as a set-up structure), and/or one or more servo controlled links (e.g. one more links that may be controlled in response to commands from the control system), and a manipulator. The manipulator assembly 102 may optionally include a plurality of actuators or motors that drive inputs on medical instrument 104 in response to commands from the control system (e.g., a control system 112). The actuators may optionally include drive systems that when coupled to medical instrument 104 may advance medical instrument 104 into a naturally or surgically created anatomic orifice. Other drive systems may move the distal end of medical instrument 104 in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulable end effector of medical instrument 104 for grasping tissue in the jaws of a biopsy device and/or the like. Actuator position sensors such as resolvers, encoders, potentiometers, and other mechanisms may provide sensor data to medical system 100 describing the rotation and orientation of the motor shafts. This position sensor data may be used to determine motion of the objects manipulated by the actuators.

Medical system 100 may include a sensor system 108 with one or more sub-systems for receiving information about the instruments of manipulator assembly 102. Such sub-systems may include a position/location sensor system (e.g., an electromagnetic (EM) sensor system); a shape sensor system for determining the position, orientation, speed, velocity, pose, and/or shape of a distal end and/or of one or more segments along a flexible body that may make up medical instrument 104; and/or a visualization system for capturing images from the distal end of medical instrument 104.

Medical system 100 also includes a display system 110 for displaying an image or representation of the clinical site and medical instrument 104 generated by sub-systems of sensor system 108. Display system 110 and master assembly 106 may be oriented so operator O can control medical instrument 104 and master assembly 106 with the perception of telepresence.

In some embodiments, medical instrument 104 may include components of an imaging system that records a concurrent or real-time image of a clinical site and provides the image to the operator or operator O through one or more displays of medical system 100, such as one or more displays of display system 110. The imaging system may be implemented as hardware, firmware, software or a combination thereof which interact with or are otherwise executed by one or more computer processors, which may include the processors of a control system 112.

Display system 110 may also display an image of the clinical site and medical instruments captured by the visualization system. In some examples, medical system 100 may configure medical instrument 104 and controls of master assembly 106 such that the relative positions of the medical instruments are similar to the relative positions of the eyes and hands of operator O. In this manner operator O can manipulate medical instrument 104 and the hand control as if viewing the workspace in substantially true presence.

Medical system 100 may also include control system 112. Control system 112 includes at least one memory and at least one computer processor (not shown) for effecting control between medical instrument 104, master assembly 106, sensor system 108, and display system 110. Control system 112 also includes programmed instructions (e.g., a non-transitory machine-readable medium storing the instructions) to implement some or all of the methods described in accordance with aspects disclosed herein, including instructions for providing information to display system 110. While control system 112 is shown as a single block in the simplified schematic of FIG. 1, the system may include two or more data processing circuits with one portion of the processing optionally being performed on or adjacent to manipulator assembly 102, another portion of the processing being performed at master assembly 106, and/or the like. The processors of control system 112 may execute instructions comprising instruction corresponding to processes disclosed herein and described in more detail below. Any of a wide variety of centralized or distributed data processing architectures may be employed. Similarly, the programmed instructions may be implemented as a number of separate programs or subroutines, or they may be integrated into a number of other aspects of the robotic systems described herein. In one embodiment, control system 112 supports wireless communication protocols such as Bluetooth, IrDA, HomeRF, IEEE 802.11, DECT, and Wireless Telemetry.

In some embodiments, control system 112 may receive force and/or torque feedback from medical instrument 104. Responsive to the feedback, control system 112 may transmit signals to master assembly 106. In some examples, control system 112 may transmit signals instructing one or more actuators of manipulator assembly 102 to move medical instrument 104. Medical instrument 104 may extend into an internal clinical site within the body of patient P via openings in the body of patient P. Any suitable conventional and/or specialized actuators may be used. In some examples, the one or more actuators may be separate from, or integrated with, manipulator assembly 102. In some embodiments, the one or more actuators and manipulator assembly 102 are provided as part of a teleoperational cart positioned adjacent to patient P and operating table T.

Control system 112 may optionally further include a virtual visualization system to provide navigation assistance to operator O when controlling medical instrument 104 during an image-guided medical procedure. Virtual navigation using the virtual visualization system may be based upon reference to an acquired preoperative or intraoperative dataset of anatomic passageways. The virtual visualization system processes images of the clinical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. Software, which may be used in combination with manual inputs, is used to convert the recorded images into segmented two dimensional or three dimensional composite representation of a partial or an entire anatomic organ or anatomic region. An image data set is associated with the composite representation. The composite representation and the image data set describe the various locations and shapes of the passageways and their connectivity. The images used to generate the composite representation may be recorded preoperatively or intra-operatively during a clinical procedure. In some embodiments, a virtual visualization system may use standard representations (i.e., not patient specific) or hybrids of a standard representation and patient specific data. The composite representation and any virtual images generated by the composite representation may represent the static posture of a deformable anatomic region during one or more phases of motion (e.g., during an inspiration/expiration cycle of a lung).

During a virtual navigation procedure, sensor system 108 may be used to compute an approximate location of medical instrument 104 with respect to the anatomy of patient P. The location can be used to produce both macro-level (external) tracking images of the anatomy of patient P and virtual internal images of the anatomy of patient P. The system may implement one or more electromagnetic (EM) sensor, fiber optic sensors, and/or other sensors to register and display a medical implement together with preoperatively recorded surgical images, such as those from a virtual visualization system. For example, PCT Publication WO 2016/191298 (published Dec. 1, 2016) (disclosing “Systems and Methods of Registration for Image Guided Surgery”) and U.S. patent application Ser. No. 13/107,562 (filed May 13, 2011) (disclosing “Medical System Providing Dynamic Registration of a Model of an Anatomic Structure for Image-Guided Surgery”), each of which is incorporated by reference herein in its entirety, discloses such systems. Medical system 100 may further include optional operations and support systems (not shown) such as illumination systems, steering control systems, irrigation systems, and/or suction systems. In some embodiments, medical system 100 may include more than one manipulator assembly and/or more than one master assembly. The exact number of teleoperational manipulator assemblies will depend on the medical procedure and the space constraints within the operating room, among other factors. Master assembly 106 may be collocated or they may be positioned in separate locations. Multiple master assemblies allow more than one operator to control one or more teleoperational manipulator assemblies in various combinations. A robotic system of the medical system 100 may comprise the manipulator assembly 102, the control system 112, and the sensor system 108. In some arrangements, the robotic system may also comprise the display system 110 and the master assembly 106.

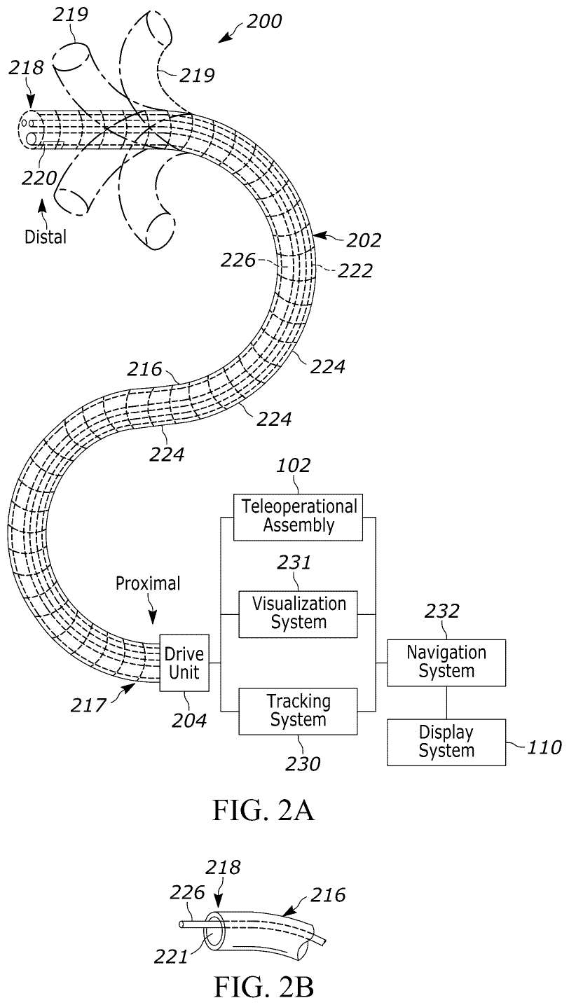

FIG. 2A is a simplified diagram of a medical instrument system 200 according to some embodiments. In some embodiments, medical instrument system 200 may be used as medical instrument 104 in an image-guided medical procedure performed with medical system 100. In some examples, medical instrument system 200 may be used for non-teleoperational exploratory procedures or in procedures involving traditional manually operated medical instruments, such as endoscopy. Optionally medical instrument system 200 may be used to gather (i.e., measure) a set of data points corresponding to locations within anatomic passageways of a patient, such as patient P.

Medical instrument system 200 includes elongate device 202, such as a flexible catheter, coupled to a drive unit 204. Elongate device 202 includes a flexible body 216 having proximal end 217 and distal end or tip portion 218. In some embodiments, flexible body 216 has an approximately 3 mm outer diameter. Other flexible body outer diameters may be larger or smaller.

Medical instrument system 200 further includes a tracking system 230 for determining the position, orientation, speed, velocity, pose, and/or shape of distal end 218 and/or of one or more segments 224 along flexible body 216 using one or more sensors and/or imaging devices as described in further detail below. The entire length of flexible body 216, between distal end 218 and proximal end 217, may be effectively divided into segments 224. If medical instrument system 200 is consistent with medical instrument 104 of a medical system 100, tracking system 230 may optionally be implemented as hardware, firmware, software or a combination thereof which interact with or are otherwise executed by one or more computer processors, which may include the processors of control system 112 in FIG. 1.

Tracking system 230 may optionally track distal end 218 and/or one or more of the segments 224 using a shape sensor 222. Shape sensor 222 may optionally include an optical fiber aligned with flexible body 216 (e.g., provided within an interior channel (not shown) or mounted externally). In one embodiment, the optical fiber has a diameter of approximately 200 μm. In other embodiments, the dimensions may be larger or smaller. The optical fiber of shape sensor 222 forms a fiber optic bend sensor for determining the shape of flexible body 216. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389 (filed Jul. 13, 2005) (disclosing “Fiber optic position and shape sensing device and method relating thereto”); U.S. patent application Ser. No. 12/047,056 (filed on Jul. 16, 2004) (disclosing “Fiber-optic shape and relative position sensing”); and U.S. Pat. No. 6,389,187 (filed on Jun. 17, 1998) (disclosing “Optical Fibre Bend Sensor”), which are all incorporated by reference herein in their entireties. Sensors in some embodiments may employ other suitable strain sensing techniques, such as Rayleigh scattering, Raman scattering, Brillouin scattering, and Fluorescence scattering. In some embodiments, the shape of the elongate device may be determined using other techniques. For example, a history of the distal end pose of flexible body 216 can be used to reconstruct the shape of flexible body 216 over the interval of time. In some embodiments, tracking system 230 may optionally and/or additionally track distal end 218 using a position sensor system 220. Position sensor system 220 may be a component of an EM sensor system with position sensor system 220 including one or more conductive coils that may be subjected to an externally generated electromagnetic field. Each coil of the EM sensor system then produces an induced electrical signal having characteristics that depend on the position and orientation of the coil relative to the externally generated electromagnetic field. In some embodiments, position sensor system 220 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point. Further description of a position sensor system is provided in U.S. Pat. No. 6,380,732 (filed Aug. 11, 1999) (disclosing “Six-Degree of Freedom Tracking System Having a Passive Transponder on the Object Being Tracked”), which is incorporated by reference herein in its entirety.

In some embodiments, tracking system 230 may alternately and/or additionally rely on historical pose, position, or orientation data stored for a known point of an instrument system along a cycle of alternating motion, such as breathing. This stored data may be used to develop shape information about flexible body 216. In some examples, a series of positional sensors (not shown), such as electromagnetic (EM) sensors similar to the sensors in position sensor 220 may be positioned along flexible body 216 and then used for shape sensing. In some examples, a history of data from one or more of these sensors taken during a procedure may be used to represent the shape of elongate device 202, particularly if an anatomic passageway is generally static.

Flexible body 216 includes a channel 221 sized and shaped to receive a medical instrument 226. FIG. 2B is a simplified diagram of flexible body 216 with medical instrument 226 extended according to some embodiments. In some embodiments, medical instrument 226 may be used for procedures such as surgery, biopsy, ablation, illumination, irrigation, or suction. Medical instrument 226 can be deployed through channel 221 of flexible body 216 and used at a target location within the anatomy. Medical instrument 226 may include, for example, image capture probes, biopsy instruments, laser ablation fibers, and/or other surgical, diagnostic, or therapeutic tools. Medical tools may include end effectors having a single working member such as a scalpel, a blunt blade, an optical fiber, an electrode, and/or the like. Other end effectors may include, for example, forceps, graspers, scissors, clip appliers, and/or the like. Other end effectors may further include electrically activated end effectors such as electrosurgical electrodes, transducers, sensors, and/or the like. In various embodiments, medical instrument 226 is a biopsy instrument, which may be used to remove sample tissue or a sampling of cells from a target anatomic location. Medical instrument 226 may be used with an imaging instrument (e.g., an image capture probe) also within flexible body 216. In various embodiments, medical instrument 226 may itself be an imaging instrument (e.g., an image capture probe) that includes a distal portion with a stereoscopic or monoscopic camera at or near distal end 218 of flexible body 216 for capturing images (including video images) that are processed by a imaging system 231 for display and/or provided to tracking system 230 to support tracking of distal end 218 and/or one or more of the segments 224. The imaging instrument may include a cable coupled to the camera for transmitting the captured image data. In some examples, the imaging instrument may be a fiber-optic bundle, such as a fiberscope, that couples to imaging system 231. The imaging instrument may be single or multi-spectral, for example capturing image data in one or more of the visible, infrared, and/or ultraviolet spectrums. Alternatively, medical instrument 226 may itself be the image capture probe. Medical instrument 226 may be advanced from the opening of channel 221 to perform the procedure and then retracted back into the channel when the procedure is complete. Medical instrument 226 may be removed from proximal end 217 of flexible body 216 or from another optional instrument port (not shown) along flexible body 216.

Medical instrument 226 may additionally house cables, linkages, or other actuation controls (not shown) that extend between its proximal and distal ends to controllably bend the distal end of medical instrument 226. Steerable instruments are described in detail in U.S. Pat. No. 7,316,681 (filed on Oct. 4, 2005) (disclosing “Articulated Surgical Instrument for Performing Minimally Invasive Surgery with Enhanced Dexterity and Sensitivity”) and U.S. patent application Ser. No. 12/286,644 (filed Sep. 30, 2008) (disclosing “Passive Preload and Capstan Drive for Surgical Instruments”), which are incorporated by reference herein in their entireties.

Flexible body 216 may also house cables, linkages, or other steering controls (not shown) that extend between drive unit 204 and distal end 218 to controllably bend distal end 218 as shown, for example, by broken dashed line depictions 219 of distal end 218. In some examples, at least four cables are used to provide independent “up-down” steering to control a pitch of distal end 218 and “left-right” steering to control a yaw of distal end 218. Steerable elongate devices are described in detail in U.S. patent application Ser. No. 13/274,208 (filed Oct. 14, 2011) (disclosing “Catheter with Removable Vision Probe”), which is incorporated by reference herein in its entirety. In embodiments in which medical instrument system 200 is actuated by a teleoperational assembly, drive unit 204 may include drive inputs that removably couple to and receive power from drive elements, such as actuators, of the teleoperational assembly. In some embodiments, medical instrument system 200 may include gripping features, manual actuators, or other components for manually controlling the motion of medical instrument system 200. Elongate device 202 may be steerable or, alternatively, the system may be non-steerable with no integrated mechanism for operator control of the bending of distal end 218. In some examples, one or more lumens, through which medical instruments can be deployed and used at a target surgical location, are defined in the walls of flexible body 216.

In some embodiments, medical instrument system 200 may include a flexible bronchial instrument, such as a bronchoscope or bronchial catheter, for use in examination, diagnosis, biopsy, or treatment of a lung. Medical instrument system 200 is also suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from tracking system 230 may be sent to a navigation system 232 where it is combined with information from imaging system 231 and/or the preoperatively obtained models to provide the operator with real-time position information. In some examples, the real-time position information may be displayed on display system 110 of FIG. 1 for use in the control of medical instrument system 200. In some examples, control system 112 of FIG. 1 may utilize the position information as feedback for positioning medical instrument system 200. Various systems for using fiber optic sensors to register and display a surgical instrument with surgical images are provided in PCT Publication WO 2016/191298 (published Dec. 1, 2016) (disclosing “Systems and Methods of Registration for Image Guided Surgery”) and U.S. patent application Ser. No. 13/107,562 (filed May 13, 2011) (disclosing “Medical System Providing Dynamic Registration of a Model of an Anatomic Structure for Image-Guided Surgery”), each of which is incorporated by reference herein in its entirety.

In some examples, medical instrument system 200 may be teleoperated within medical system 100 of FIG. 1. In some embodiments, manipulator assembly 102 of FIG. 1 may be replaced by direct operator control. In some examples, the direct operator control may include various handles and operator interfaces for hand-held operation of the instrument.

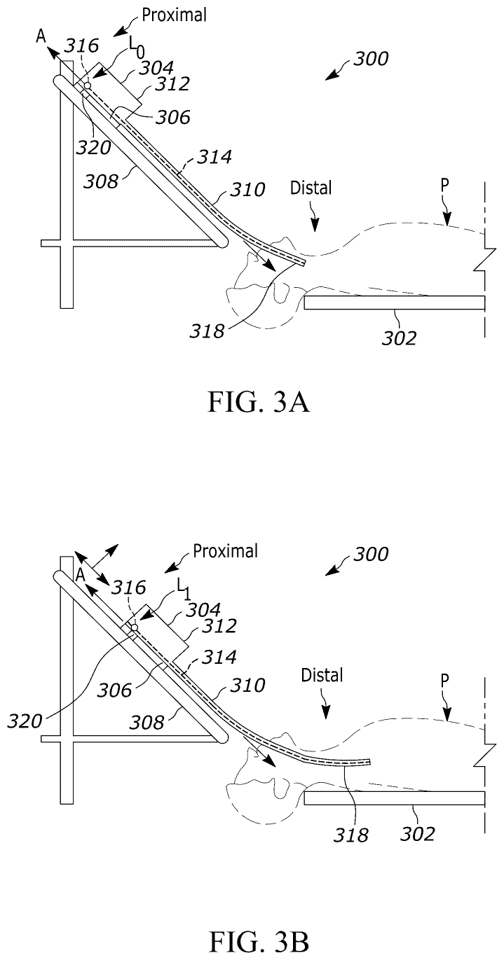

FIGS. 3A and 3B are simplified diagrams of side views of a patient coordinate space including a medical instrument mounted on an insertion assembly according to some embodiments. As shown in FIGS. 3A and 3B, a surgical environment 300 includes a patient P positioned on the table T of FIG. 1. Patient P may be stationary within the surgical environment in the sense that gross patient movement is limited by sedation, restraint, and/or other means. Cyclic anatomic motion including respiration and cardiac motion of patient P may continue, unless patient is asked to hold his or her breath, or mechanical ventilation is paused, to temporarily suspend respiratory motion. Accordingly, in some embodiments, data may be gathered at a specific, phase in respiration, and tagged and identified with that phase. In some embodiments, the phase during which data is collected may be inferred from physiological information collected from patient P. Within surgical environment 300, a point gathering instrument 304 is coupled to an instrument carriage 306. In some embodiments, point gathering instrument 304 may use EM sensors, shape-sensors, and/or other sensor modalities. Instrument carriage 306 is mounted to an insertion stage 308 fixed within surgical environment 300. Alternatively, insertion stage 308 may be movable but have a known location (e.g., via a tracking sensor or other tracking device) within surgical environment 300. Instrument carriage 306 may be a component of a manipulator assembly (e.g., manipulator assembly 102) that couples to point gathering instrument 304 to control insertion motion (i.e., motion along the A axis) and, optionally, motion of a distal end 318 of an elongate device 310 in multiple directions including yaw, pitch, and roll. Instrument carriage 306 or insertion stage 308 may include actuators, such as servomotors, (not shown) that control motion of instrument carriage 306 along insertion stage 308.

Elongate device 310 (which may be substantially similar to elongate device 202 described with respect to FIG. 2 above) is coupled to an instrument body 312. As shown in FIG. 3, instrument body 312 is coupled and fixed relative to instrument carriage 306. In some embodiments, an optical fiber shape sensor 314 is fixed at a proximal point 316 on instrument body 312. In some embodiments, proximal point 316 of optical fiber shape sensor 314 may be movable along with instrument body 312 but the location of proximal point 316 may be known (e.g., via a tracking sensor or other tracking device). Shape sensor 314 measures a shape from proximal point 316 to another point such as distal end 318 of elongate device 310. Point gathering instrument 304 may be substantially similar to medical instrument system 200 described with respect to FIG. 2 above.

Returning to FIG. 3, a position measuring device 320 provides information about the position of instrument body 312 as it moves on insertion stage 308 along an insertion axis A. Position measuring device 320 may include resolvers, encoders, potentiometers, and/or other sensors that determine the rotation and/or orientation of the actuators controlling the motion of instrument carriage 306 and consequently the motion of instrument body 312. In some embodiments, insertion stage 308 is linear. In some embodiments, insertion stage 308 may be curved or have a combination of curved and linear sections.

FIG. 3A shows instrument body 312 and instrument carriage 306 in a retracted position along insertion stage 308. In this retracted position, proximal point 316 is at a position L0 on axis A. In this position along insertion stage 308, a component of the location of proximal point 316 may be set to a zero and/or another reference value to provide a base reference to describe the position of instrument carriage 306, and thus proximal point 316, on insertion stage 308. With this retracted position of instrument body 312 and instrument carriage 306, distal end 318 of elongate device 310 may be positioned just inside an entry orifice of patient P. An airway management device, such as airway management device 500 discussed below, may be inserted in the patient's trachea through the patient's mouth to provide access to the patient's anatomy for the distal end 318 of the instrument body 312. Optionally, the airway management device 500 may be releasably coupled with the insertion stage 308. Also in this position, position measuring device 320 may be set to a zero and/or another reference value (e.g., I=0). In FIG. 3B, instrument body 312 and instrument carriage 306 have advanced along the linear track of insertion stage 308 and distal end 318 of elongate device 310 has advanced into patient P. In this advanced position, the proximal point 316 is at a position L1 on the axis A. In some examples, encoder and/or other position data from one or more actuators controlling movement of instrument carriage 306 along insertion stage 308 and/or one or more position sensors associated with instrument carriage 306 and/or insertion stage 308 is used to determine the position Lx of proximal point 316 relative to position L0. In some examples, position Lx may further be used as an indicator of the distance or insertion depth to which distal end 318 of elongate device 310 is inserted into the passageways of the anatomy of patient P.

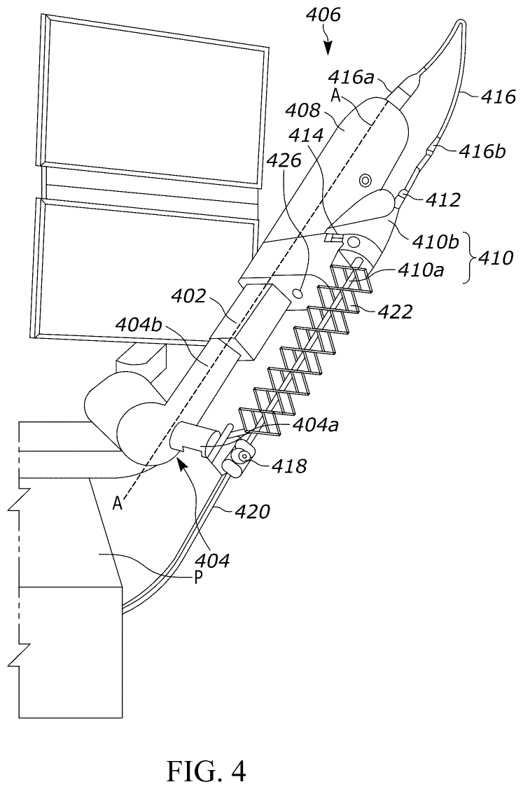

FIG. 4 shows a detailed example of an instrument manipulator 406, which may be used as manipulator assembly 102 of the robotic system in FIG. 1. The instrument manipulator 406 may include a base 404, an insertion stage 402 (which may be substantially similar to insertion stage 308 of FIGS. 3A and 3B), and an instrument carriage 408 (which may be substantially similar to instrument carriage 306 of FIGS. 3A and 3B) to which a flexible elongate device 410 (which may be substantially similar to elongate device 202 of FIG. 2 or elongate device 310 of FIG. 3) is coupled. In one or more embodiments, the instrument manipulator 406 shown in FIG. 4 provides for insertion and retraction of the flexible elongate device 410, with respect to the patient anatomy, by moving the instrument carriage 408 and insertion stage 402 in a telescoping manner relative to the base 404 and along the linear axis A. The instrument manipulator 406, thus, provides an insertion degree of freedom for the insertion and retraction of the flexible body portion 410a along the linear axis A. In a medical scenario, the insertion may advance the flexible body portion 410a into the patient anatomy, whereas the retraction may withdraw the flexible body portion 410a from the patient anatomy.

The base 404 includes a shaft portion 404a and a main portion 404b. As described in detail below, the shaft portion 404a removably couples to a connection mechanism 418 which receives the flexible body portion 410a. The insertion stage 402 is coupled to the main portion 404b of the base 404 and translates along the main portion 404b. The instrument carriage 408 is coupled to and translates along the insertion stage 402. The flexible elongate device 410 may include a flexible body portion 410a and a control assembly 410b. The instrument carriage 408 couples to the control assembly 410b at an instrument interface 414 of the instrument carriage 408. The instrument manipulator 406 also couples to a probe assembly 416 which includes a probe 416b and a probe connector 416a. The probe assembly 416 may insert into a working lumen of the flexible body portion 410a through a connector 412 on the control assembly 410b and may run through the flexible body portion 410a. The probe 416b may include, for example, a viewing scope assembly that provides images of a clinical site. The instrument carriage 408 may include electronic and optical components providing probe 416b with endoscopic capabilities. In some embodiments, the probe assembly 416 may be detached from the instrument manipulator 406 and control assembly 410b, and removed from the flexible elongate device 410. Alternative instruments such as biopsy needles, ablation tools, and other flexible instruments may be coupled to the instrument manipulator 406 and/or the flexible elongate device 410, through the flexible body portion 410a working lumen.

Continuing with FIG. 4, the connection mechanism 418 may include a manipulator interface which may be removably coupled to the base 404, a distal end which may be removably coupled to a patient medical device 420, e.g., an airway management device, and a proximal end which may receive the flexible body portion 410a. The patient medical device 420 (e.g., an endotracheal tube, a laryngeal mask airway, a cannula, etc.) may be fixed to the patient anatomy to facilitate insertion of various medical devices into the patient anatomy. For example, the patient medical device 420 may be a laryngeal mask airway 500 as discussed further below. Various systems and methods related to connection mechanisms are described in PCT/US2018/017085 (filed Feb. 6, 2018) (disclosing “Systems and Methods for Coupling Components of a Medical System”), which is incorporated by reference herein in its entirety. In some embodiments, the flexible body portion 410a runs through a flexible elongate device guide 422, which is a selectively collapsible and extendable device that supports the length of the flexible body portion 410a during movement of the instrument carriage 408. The flexible body portion 410a without guidance may buckle in regions with no lateral support, e.g., in the space between the instrument interface 414 and the connection mechanism 418. To avoid the buckling, the flexible elongate device guide 422 may operate as a flexible elongate device guide by providing lateral support to the flexible body portion 410a proximal to the patient. Various systems and methods related to flexible elongate device guides are described in PCT/US2017/041160 (filed Jul. 7, 2017) (disclosing “Guide Apparatus for Delivery of an Elongate Device and Methods of Use”), which is incorporated by reference herein in its entirety.

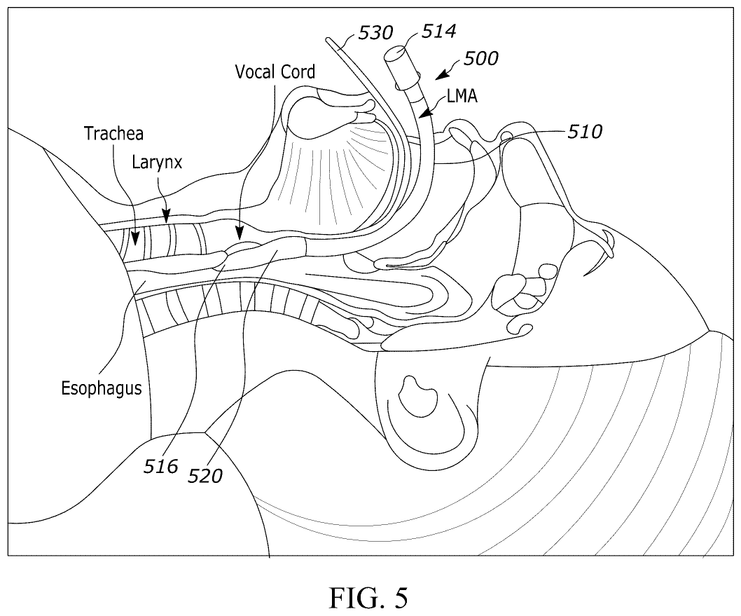

FIG. 5 illustrates an example of an airway management device 500 that may be used as the patient medical device 420 in conjunction with the instrument manipulator 406 described above. Specifically, FIG. 5 illustrates an LMA, inserted into the patient's body through the patient's mouth while the patient lies on their back with the neck slightly extended using, for example, the instrument manipulator 406 discussed above. The airway management device 500 includes an elongated, flexible, and hollow tube 510 curved between its distal end 516 and proximal end 514 for insertion through the upper airway passages. The proximal end 514 of the hollow tube 510 is also the proximal end 514 of the airway management device 500. The airway management device 500 may also include an inflatable balloon-like structure or cuff 520 disposed at the distal end 516 of the airway management device 500 that is inflated using a cuff-inflating tube 530. This balloon-like structure or cuff 520 forms an airtight seal on proximal to the larynx and the trachea, thereby preventing air being pumped by a ventilator/breathing machine connected to the proximal end of the tube 510 from escaping backward through the trachea 540 and entering the oral and nasal passages. As shown, the airway management device 500 is not placed within the trachea of the patient.

To avoid trauma to the patient due to expected or unexpected patient motion during the medical procedure and/or to avoid dislodgement of the airway management device 500 from the patient's body, a connection mechanism between the instrument manipulator 406 of the robotic system (e.g., medical system 100 of FIG. 1) and the airway management device 500 is configured to move in various degrees of freedom to accommodate for the expected and unexpected patient motion. In cases where the patient motion causes a significant amount of displacement, and therefore force on the connection mechanism between the robotic system and the airway management device 500, the connection mechanism may be configured to decouple from either the robotic system or from the airway management device. The decoupling mechanisms may be purely mechanical or may include sensors to sense the forces on the connection, and decouple, when necessary, the connection when the forces exceed a predetermined threshold to ensure patient safety. Alternatively, patient motion may be sensed using sensors coupled to the patient.

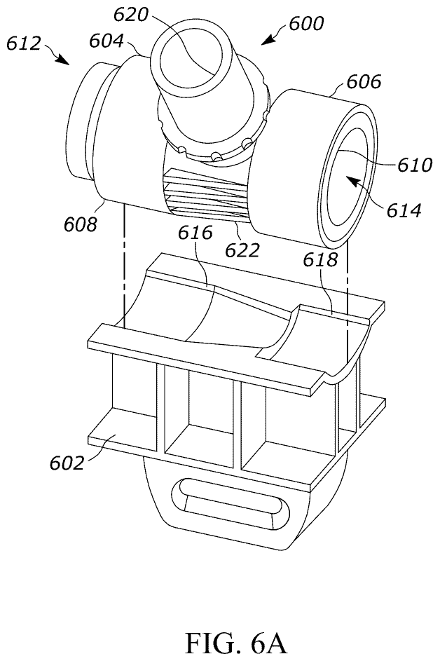

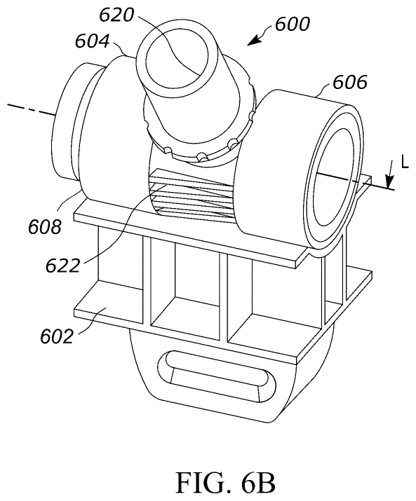

An example of a suitable connection mechanism for coupling the airway management device 500 with the instrument manipulator 406 or other component of a robotic system is described with reference to the following figures, FIGS. 6A and 6B, which illustrate an exemplary version of a connection mechanism 600. The connection mechanism 600 may rotatably (e.g., relative to an axis) couple to a mating bracket 602, which may be integrated into a docking spar of a flexible manipulator assembly 460 or other component of a robotic system. More specifically, the connection mechanism 600 includes cylindrical coupling members 604 and 606 extending on opposite ends of a connector body 608 located at a medial portion of the connection mechanism 600. A passage 610 extends through the body 608. An end 612 of the connection mechanism 600 may couple to an elongate device (e.g., elongate device 410), and a second end 614 of the connection mechanism 600 may couple to an airway management device (e.g., airway management device 311 and/or 478).

A first end portion may include the coupling members 604 and 606. The members 604 and 606 have curved (e.g., cylindrical, toroidal, partially spherical, etc.) exterior surfaces to mate with curved surfaces 616 and 618, respectively, of the bracket 602, ultimately coupling to the robotic system. The coupling members 604 and 606 may be retained magnetically, and accordingly, the members 604 and 606 and the bracket 602 may each include magnets and/or a material responsive to a magnetic field. In some such examples, the bracket 602 include magnets (e.g., permanent magnets, electromagnets, hybrid magnets, etc.) proximate to the curved surfaces 616 and 618, while the coupling members 604 and 606 include magnets or a material responsive to a magnetic field (e.g., iron, nickel, cobalt, a ferritic compound, etc.), or vice-versa.

When magnetically attached, as shown in FIG. 6B, the coupling members 604 and 606 may rotate about the longitudinal axis L, with respect to the bracket 602, while the body 608 remains laterally coupled to the bracket. The amount of rotation may be limited by contact with the bracket 602 or the bracket 602 may permit the connection mechanism 600 to rotate a full 360°. Accordingly, in various embodiments, the connection mechanism 600 may rotate between about 180° and about 360°. In other embodiments, the connection mechanism 600 can be configured to rotate less than 180°. This rotation may occur in response to slight movement of the patient or manipulator assembly 460. A tube 620 couples the connection mechanism 600 to a source of air and/or anesthesia. The magnetic connection allows free rotation of the connector in response to forces from the air and anesthesia tubing. With the connection mechanism 600 attached to the airway management device 500 (shown in FIG. 5), patient movement greater than a threshold may generate a force that causes the release of the magnetic members 604 and 606 from the bracket 602. Thus, the connection mechanism 600 coupled to the airway management device 500 (all attached to the patient) separates from the bracket 602. The magnets of the members 604 and 606 may be selected to release in response to a predetermined force or motion.

Optionally, the connection mechanism 600 and/or the bracket 602 may include Hall sensors to detect when the connection mechanism is completely seated, partially seated, or not seated to the bracket 602. Various control modes of the robotic system may be activated depending on the detected seating of the connection mechanism 600. Optionally, the connection mechanism 600 may include a set of fins 622 which create a tapered body profile that provides assistance with the directional mounting (i.e., prevents upside-down mounting). As shown in FIG. 6A, the curved surface 616 is tapered to mate with a corresponding tapered body profile of the connection mechanism 600.

In some embodiments, the portions of the connection mechanism 600 that couple to the air source, the elongate device, and/or the endotracheal tube rotate or permit rotation relative to the connection mechanism 600 to make the connections more compliant and to make it easier to complete the connections. In one such example, the tube 620 providing a direct coupling to the source of air and/or anesthesia rotates and/or permits rotation of the coupled source. In one such example, the first end 612 of the connection mechanism 600 that couples to the elongate device and the second end 614 of the connection mechanism 600 that couples to the airway management device rotate and/or permit rotation of the coupled device. This may also prevent the coupled devices from inadvertently causing the connection mechanism 600 to release from the bracket 602 or from inadvertently preventing the connection mechanism 600 from releasing.

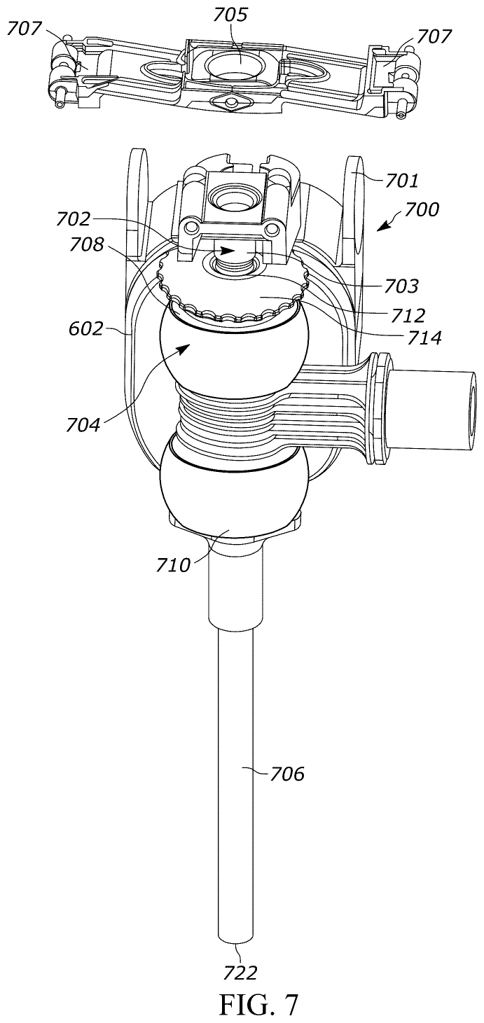

When the airway management device 500 terminates proximally to a patient's trachea, for example if an LMA is being used, an apparatus may be beneficial to provide structural support for moving a flexible elongate device (e.g., elongate device 202 or 310 or flexible body portion 410a) to a target location. As described above in the background section above, in the absence of such structural support, the flexible elongate device can prolapse (e.g., have an instability, such as buckling, collapsing, weakening, etc.) and be unable to reach a target destination. The apparatus disclosed herein can function as both a connection mechanism substantially similar to connection mechanism 600 described above and can further secure an overtube that provides the requisite structural support. FIGS. 7-15 illustrate such an apparatus 700.

The apparatus 700 movably couples the robotic system (e.g., medical system 100) to the airway management device (e.g., 420 or 500). As shown in FIG. 7, the apparatus 700 includes an overtube 702 and a connection mechanism 704 substantially similar to connection mechanism 600 but modified to connect with the overtube 702. The overtube 702 includes a flexible tubular portion 706. The connection mechanism includes a mating bracket or docking spar 602. The connection mechanism 704 includes a first portion 708 configured to receive the overtube 702). The connection mechanism 704 further includes a second portion 710 configured to be connected with a proximal end of an airway management device (e.g., proximal end 514 of airway management device 500 of FIG. 5). A lumen 712 is defined within the proximal portion 708 and the second portion 710. A locking mechanism 714 is configured to connect to the flexible tubular portion 706 of the overtube 702. When the overtube 702 is connected with the connection mechanism 704, the flexible tubular portion 706 of the overtube 702 is within the lumen 712 of the connection mechanism 704 and may extend distal to the distal end of the airway management device (e.g., distal end 516 of the airway management device 500 of FIG. 5). A proximal end portion 703 of the overtube 702 is wider than the width of the lumen 712 and thereby constrains the distal movement of the overtube 702 through the connection mechanism 704.

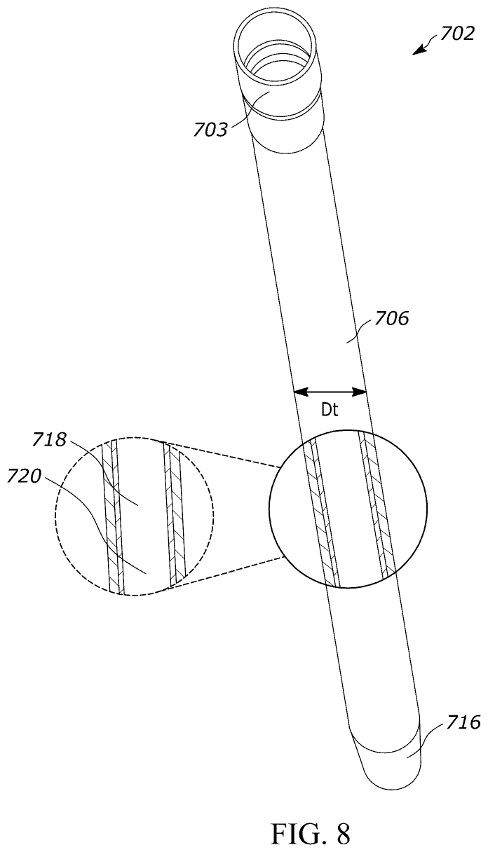

The robotic system (e.g., system components of medical system 100 of FIG. 1) to which the apparatus 700 connects is configured to drive a flexible elongate device (e.g., elongate device 202 of FIG. 2). The airway management device (e.g., airway management device 500 of FIG. 5) to which the apparatus 700 connects includes a cylindrical passageway (e.g., hollow tube 510) configured to couple with the robotic system via the connection mechanism. The airway management device further includes an engagement portion (e.g., cuff 520) attached to the cylindrical passageway that is configured to engage a patient anatomy proximal to the trachea. The flexible tubular portion 706 of the overtube 702 of FIG. 7 is configured to be inserted within the cylindrical passageway (e.g., hollow tube 510) of the airway management device 500 and to extend distal to the engagement portion (e.g., cuff 520) when inserted within the cylindrical passageway of the airway management device. With this configuration, the flexible tubular portion 706 of the overtube 702, shown in FIG. 8, structurally supports the flexible elongate device being driven by the robotic system when the flexible elongate device is received within flexible tubular portion 706 and provides additional support to the flexible elongate device beyond the airway management device 500.

As described with respect to FIG. 4 above, the robotic system includes an instrument carriage (e.g., instrument carriage 408) and a flexible elongate device guide (e.g., flexible elongate device guide 422) configured to provide lateral support to the flexible elongate device during movement of the instrument carriage. In the arrangement shown in FIG. 7, the flexible elongate device guide 422 includes an eyelet 705 aligned with the overtube 702 and arms 707. When used with apparatus 700, the flexible elongate device guide (e.g., flexible elongate device guide 422) is configured to accommodate the overtube 702. Because the overtube 702 must have larger outer diameter than the flexible elongate device (e.g., flexible body portion 410a) in order for the flexible elongate device to move within the flexible tubular portion 706, at least some dimensions of the flexible elongate device guide may be increased to permit passage of the overtube 702. In some arrangements, portions of the flexible elongate device guide may be modified, such as eyelet 705 and a distal connection 701, for use with the apparatus 700.

Turning to FIG. 8, the flexible tubular portion 706 of the overtube 702 may include an atraumatic tip 716. The atraumatic tip 716 ensures that the overtube 702 does not injure the patient during insertion by, for example, having a blunt or curved surface, or being made of a relatively softer material than the main portion of the overtube 702. As shown in the cutaway portion, the flexible tubular portion 706 has an interior surface 718 comprising a low friction interface that defines a lumen 722. The low friction interface enables the flexible elongate device (e.g., elongate device 202 or flexible body portion 410a) to move through the overtube 702 more easily. The flexible tubular portion diameter Dt. An end portion 703 of the overtube has a diameter greater than Dt to, as discussed above, constrain the distal movement of the overtube 702 through the connection mechanism 704.

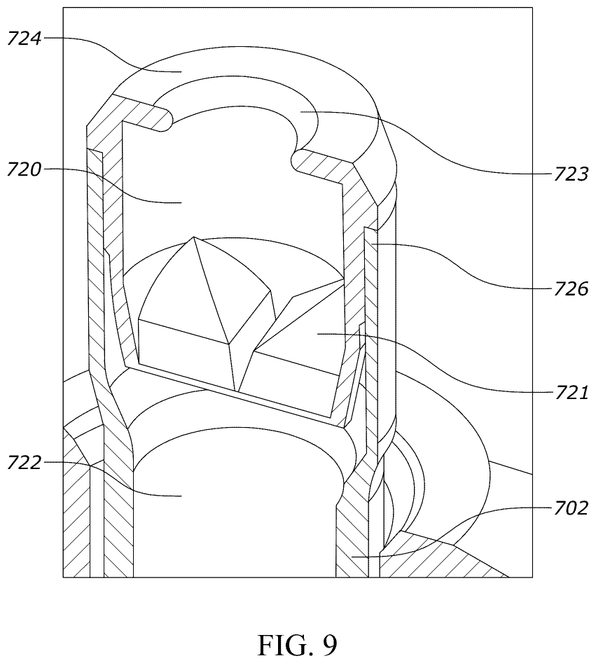

Turning to FIG. 9, the apparatus 700 further includes a first seal 720. The first seal 720 is configured to seal the lumen 722 of the flexible tubular portion 706 of the overtube 702. In the arrangement shown, the first seal 720 includes a cross-slit valve 721 and a septum 723. The septum 723 is positioned proximal to the cross-slit valve 721 so that, as a flexible elongate device is inserted, the septum 723 seals around the flexible elongate device before the cross-slit valve 721 is opened by the flexible elongate device. The first seal 720, and specifically the cross-slit valve 721, may be configured to seal the lumen 722 of the overtube 702 in the absence of the flexible elongate device within the overtube 702. The first seal 720, and specifically the septum 723, may be configured to seal around the flexible elongate device to continue to seal the lumen 722 when the flexible elongate device is inserted through the overtube 702. As shown, the seal 720 is provided on a cap 724 that is secured at a proximal end 726 of the overtube 702 and extends partially into the lumen 722 of the flexible tubular portion 706 of the overtube 702.

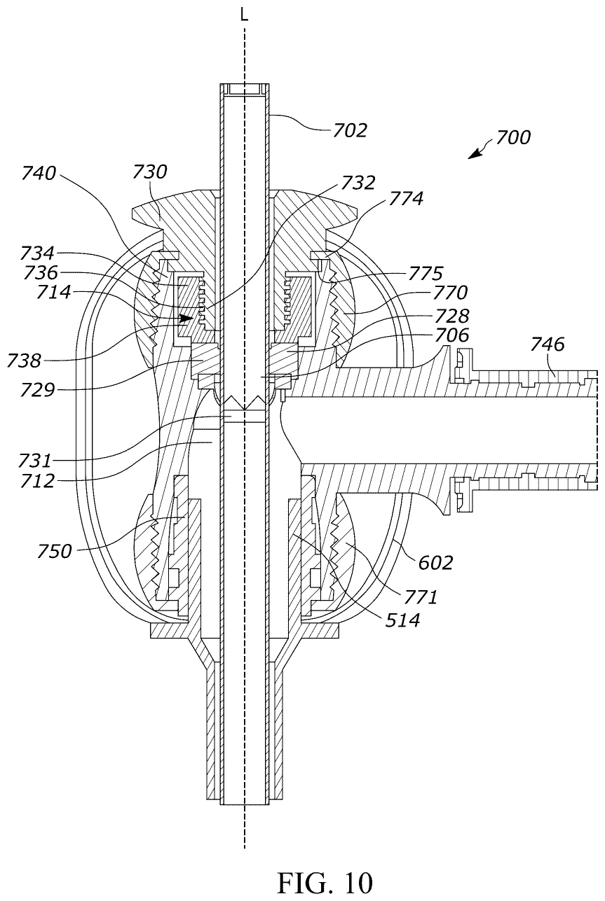

As shown in FIG. 10, the apparatus 700 further includes a second seal 728. The second seal 728 is configured to seal the lumen 712 of the connection mechanism 704. The second seal 728 may include a septum seal 729 and a cross-slit valve 731, similar to the first seal 720 depicted in FIG. 9 above but larger to accommodate the overtube 702 when inserted within the connection mechanism 704. The second seal 728, and specifically the cross-slit valve 731 may be configured to seal the lumen 712 of the connection mechanism 704 in the absence of the overtube 702 within the connection mechanism 704. The second seal 728, and specifically the septum 729, may be configured to seal around the overtube 702 to continue to seal the lumen 712 when the overtube 702 is inserted through the connection mechanism 704. Although the first seal 720 and the second seal 728 are describe as each including a cross-slit valve and a septum, other types of sealing structures may be used for each of these seals to provide for sealing and prevention of fluid transmission in both the presence and absence of inserted objects.

The locking mechanism 714 is configured to mechanically secure the overtube 702 to the connection mechanism 704. For example, the locking mechanism 714 is configured to secure the second seal 728, and specifically the septum seal 729, around the flexible tubular portion 706 to prevent movement of the overtube 702. The locking mechanism 714 includes a first lock part 730 that has a threaded base 732. The locking mechanism 714 further includes a second lock part 734 having a threaded wall 736 configured to engage the threaded base 732. The second lock part 734 also has a foot 738 that extends inwardly from the threaded wall 736. The second lock part 734 is positioned adjacent the second seal 728, and relative movement between the threaded base 732 of the first lock part 730 and the threaded wall 736 of the second lock part 734 causes the foot 738 of the second lock part 734 to compress the second seal 728, thereby causing the second seal 728 to expand radially inward to engage the flexible tubular portion 706 of the overtube 702. The locking mechanism 714 further includes an outer housing 740. The second lock part 734 includes splines 742 on an outside 744 of the threaded wall 736, as shown in FIG. 12. The splines 742 are configured to engage the outer housing 740 so that the second lock part 734 cannot rotate relative to the outer housing 740. The first lock part 730 is constrained vertically so does not move vertically as the second lock part 734 rotates relative to the first lock part 730. The threads on the first lock part 730 and the second lock part 734 cause the first lock part 730 and the second lock part 734 to separate because the second lock part 734 cannot rotate due to the splines 742. Because the first lock part 730 is constrained vertically, the second lock part 734 moves downward and compresses the second seal 728. Specifically, the first lock part 730 includes a groove 775 that engage with a lip 774 of a coupling member 770 (discussed below), and the engagement between the groove and the lip 774 constrains the first lock part 730 vertically. In alternative example where the first lock part 730 is not constrained vertically, the first lock part 730 could thread directly into the outer housing 740 to compress the second seal 728.

FIG. 10 also illustrates features of apparatus 700 that are substantially similar to or provide similar functionality as features of connection mechanism 600. For example, the connection mechanism 704 includes a tube 746 like tube 620. The tube 746 connect the apparatus 700, and the airway management device, to a ventilator, fluid source, and/or vacuum source. As commonly understood, the connections can include one or more seal members, such as o-rings. Like tube 620, the tube 746 may be rotatably coupled to allow free rotation in response to forces from the air and anesthesia tubing. The apparatus further includes coupling members 770 and 771 that, like coupling members 604 and 606, respectively, allow the connection mechanism 704 to be coupled to a manipulator assembly (such as instrument manipulator 406) in manner that allows rotation around an axis L. The apparatus 700 includes the lumen 712, similar to passage 610, that extends through the outer housing 740. While the mating bracket 602 may be magnetically coupled to the coupling members 604 and 606 in connection mechanism 600 of FIGS. 6A and 6B, for the apparatus 700, the coupling members 770 and 771 may be magnetically connected to the mating bracket 602. The coupling members 770 and 771 may include magnets (e.g., permanent magnets, electromagnets, hybrid magnets, etc.) or a material responsive to a magnetic field to allow a magnetic attachment. As a result of the coupling members 770 and 771, the connection mechanism 704 is configured to at least partially disconnect from the robotic system when motion between the robotic system and the airway management device includes an above-threshold displacement of the airway management device relative to the robotic system. The above-threshold displacement creates a force that overcomes the magnetic force securing the connection mechanism 704 to the robotic system. As described with respect to connection mechanism 600 above, this decoupling avoids trauma to the patient due to patient motion during the medical procedure and avoids dislodgment of the airway management device form the patient's body.

The apparatus 700 further includes a coupler 750 that connects to the proximal end 514 of the airway management device 500. The coupler 750 can rotate relative to the outer housing 740 and the coupling member 771.

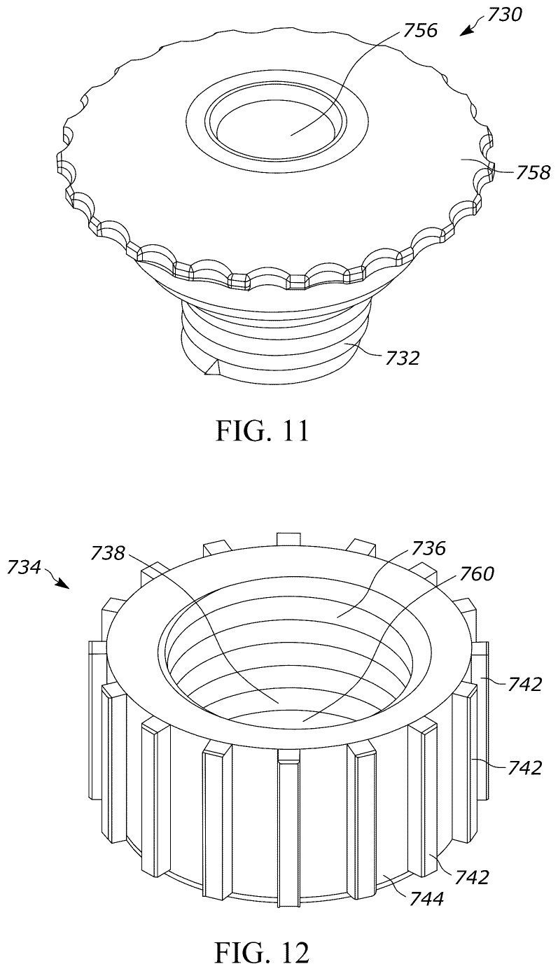

FIG. 11 illustrates the first lock part 730. As discussed above, the first lock part 730 includes a threaded base 732 to connect with the second lock part 734. The first lock part 730 further includes an aperture 756 to accommodate overtube 702 and a grippable surface 758 to facilitate relative motion between the first lock part 730 and the second lock part 734 by, for example, rotating the first lock part 730. The grippable surface 758 may include divots, textures, or other features to increase the ease with which the grippable surface can be handled.

FIG. 12 illustrates the second lock part 734. As discussed above, the second lock part 734 includes a threaded wall 736 to engage the threaded base 732 of the first lock part 730. The second lock part 734 also includes an aperture 760 to accommodate the overtube 702. The foot 738 of the second lock part 734 is adjacent to the aperture 760 and extends inwardly toward the aperture 760. The splines 742 of the second lock part 734 that prevent the second lock part 734 from rotating relative to the outer housing 740 are located on an outside 744 of the second lock part 734.

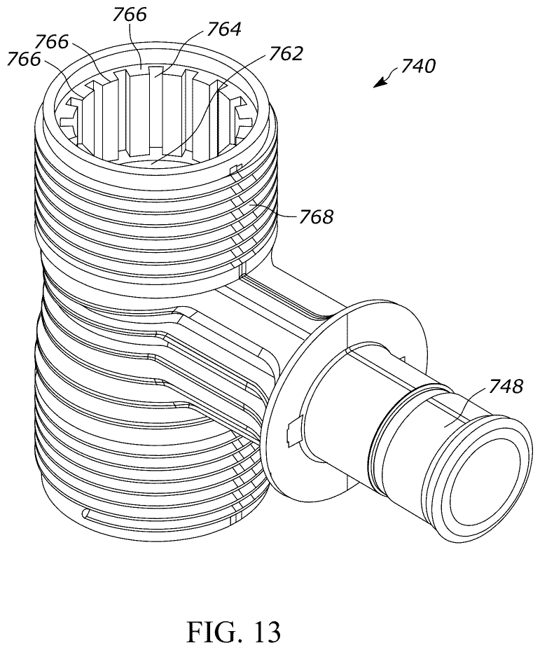

FIG. 13 illustrates the outer housing 740. A passage 762 extends through the outer housing 740. The interior surface 764 of the outer housing 740 includes splines 766 to engage the splines 742 of the second lock part 734 to prevent the second lock part 734 from rotating relative to the outer housing 740. In the arrangement shown, the outer housing 740 includes a threaded exterior surface 768.

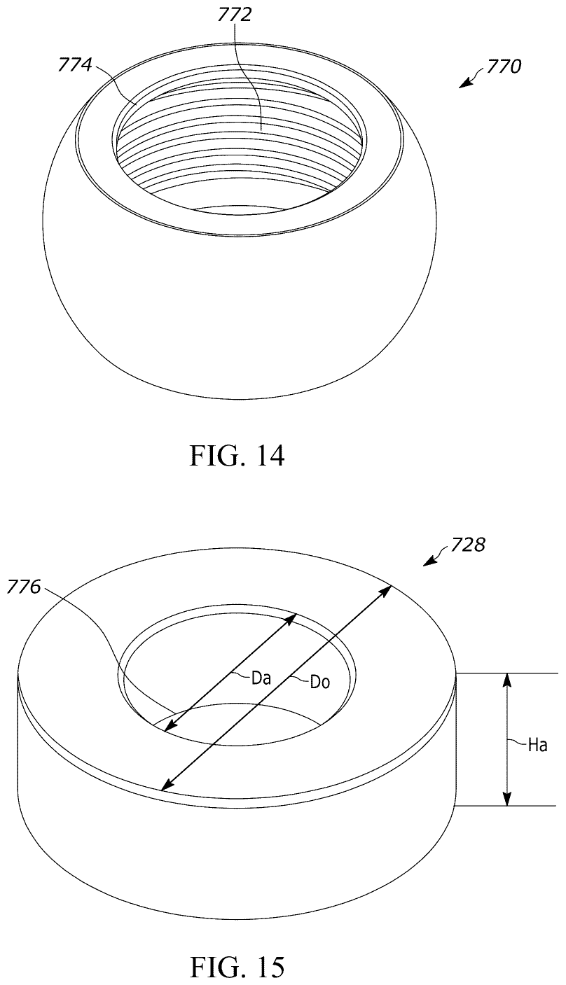

FIG. 14 illustrates the coupling member 770 with a threaded interior surface 772 to couple to the threaded exterior surface 768. As shown in FIG. 10, the coupling member 771 can include a threaded interior surface similar to that shown in FIG. 14 for the coupling member 770 to couple to the threaded exterior surface 768. The coupling member 770 also has an upper lip 774 that is positioned proximally to the outer housing 740 and extends inwardly toward the passage 762 of the outer housing 740.

FIG. 15 illustrates the second seal 728, depicted here as a septum. The second seal 728 has a height Ha and an outer diameter Do and includes a septum aperture 776 that has an inner diameter Da. As the first lock part 730 and the second lock part 734 move along the threads of the threaded base 732 and the threaded wall 736, the foot 738 of the second lock part 734 compresses the second seal 728. As the second seal 728 is compressed, the height Ha is decreased. In the absence of constraints, the diameter Da would decrease and the outer diameter Do would expand as the height Ha is decreased. However, the locking mechanism 714 constrains the second seal 728 such that the outer diameter Do expands only slightly before being limited by the locking mechanism 714, thereby causing the diameter Da to decrease more than would occur in the absence of the locking mechanism 714. Optimally, the diameter Da is sized relative to the diameter Dt of the flexible tubular portion 706 of the overtube 702 to allow insertion of the overtube 702 through the apparatus 700 prior to or with limited engagement by the foot 738 while simultaneously allowing sufficient engagement between the second seal 728 and the flexible tubular portion 706 of the overtube 702 for the second seal 728 to form a seal around the flexible tubular portion 706. The diameter Da then decreases as the foot 738 compresses the second seal 728, resulting in engagement between the second seal 728 and the overtube 702. At the same time, the slight increase in the outer diameter Do causes the second seal 728 to press against the outer housing 740, increasing the integrity of the seal.

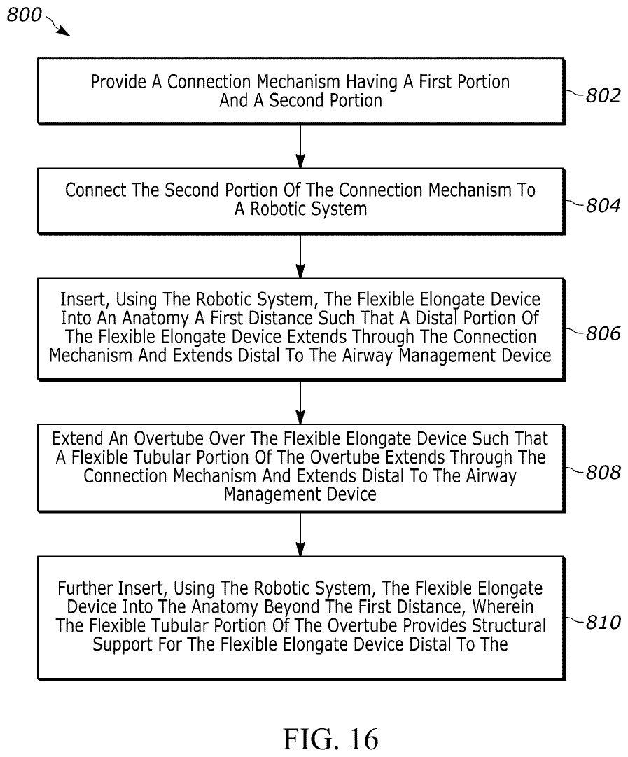

FIG. 16 illustrates a method 800 of positioning a flexible elongate device (e.g., elongate device 202). At box 802, the method 800 includes providing a connection mechanism 704 having a first portion 708 and a second portion 710. At box 804, the method 800 includes connecting the second portion 710 of the connection mechanism 704 to an airway management device 500 and then connecting to robotic system. At box 806, the method 800 includes inserting, using the robotic system, the flexible elongate device into an anatomy a first distance such that a distal portion of the flexible elongate device extends through the connection mechanism 704 and extends distal to the airway management device 500. For example, the distal portion of the flexible elongate device may extend into the trachea that is distal to the engagement portion of the airway management device. At box 808, the method 800 includes extending an overtube 702 over the flexible elongate device such that a flexible tubular portion 706 of the overtube 702 extends through the connection mechanism 704 and extends distal to the airway management device 500. In some examples, the overtube 702 is initially configured to not extend beyond the airway management device 500. In some examples, the flexible tubular portion 706 of the overtube does not extend distal to the distal portion of the flexible elongate device. This allows the flexible elongate device to serve as a guide for the entire insertion of overtube 702, thereby ensuring proper overtube insertion location and minimizing the chances of damaging the anatomy. At box 810, the method 800 includes further inserting, using the robotic system, the flexible elongate device into the anatomy beyond the first distance, wherein the flexible tubular portion 706 of the overtube 702 provides structural support for the flexible elongate device distal to the airway management device during further insertion. The method 800 may include repeating the steps described in boxes 808 through 810 for subsequent distances (a second distance, a third distance, etc.). For example, the insertion distance of the overtube within the anatomy may be adjusted as necessary to provide sufficient support for the flexible elongate device beyond the distal end of the airway management device.

The method 800 may further include locking the flexible tubular portion 706 of the overtube 702 to the connection mechanism 704. Locking the flexible tubular portion 706 of the overtube 702 to the connection mechanism may include sealing, using a second seal 728, the lumen 712 of the connection mechanism 704. The method 800 may further include causing relative movement between the threaded base 732 of the first lock part 730 and the threaded wall 736 of the second lock part 734 to cause the foot 738 of the second lock part 734 to compress the seal 728, thereby causing the seal 728 to expand radially inward to engage the flexible tubular portion 706 of the overtube 702. The method 800 may further include extending the flexible elongate device guide (e.g., flexible elongate device guide 422) when the flexible elongate device is inserted into the anatomy the first distance. The method 800 may further include retracting the flexible elongate device guide (e.g., flexible elongate device guide 422) after extending the overtube 702 over the flexible elongate device such that the flexible tubular portion 706 of the overtube 702 extends through the connection mechanism 704 and extends distal to the airway management device 500.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the foregoing description, one or more elements shown and described in association with one example, implementation, or application may be incorporated into other examples, implementations, or application unless specifically described otherwise, unless the one or more elements would make an example or implementation non-functional, or unless two or more of the elements provide conflicting functions. Similarly, it should be understood that any particular element, including a system component or a method process, is optional and is not considered to be an essential feature of the present disclosure unless expressly stated otherwise.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions provided herein are for specific examples and it is contemplated that different sizes, dimensions, and/or ratios may be utilized to implement the concepts of the present disclosure.

While some examples are provided herein in the context of medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques may also be used for surgical and nonsurgical medical treatment or diagnosis procedures.

The methods described herein are illustrated as a set of operations or processes. Not all the illustrated processes may be performed in all examples of the methods. Additionally, one or more processes that are not expressly illustrated or described may be included before, after, in between, or as part of the example processes. In some examples, one or more of the processes may be performed by the control system (e.g., control system 112) or may be implemented, at least in part, in the form of executable code stored on non-transitory, tangible, machine-readable media that when run by one or more processors (e.g., processors of control system 112) may cause the one or more processors to perform one or more of the processes.

One or more elements in examples of this disclosure may be implemented in software to execute on a processor of a computer system such as control processing system. When implemented in software, the elements of the examples of the present disclosure are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In one example, the control system supports wireless communication protocols such as Bluetooth, IrDA, HomeRF, IEEE 802.11, DECT, and Wireless Telemetry.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the present disclosure are not described with reference to any particular programming language. It will be appreciated that a variety of programming languages may be used to implement the teachings of the present disclosure.

This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). The “pitch” direction and “yaw” direction are not necessarily limited to vertical and horizontal movement, respectively, but rather may be arbitrary directions orthogonal to one another. As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along a length of an object. As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used in this specification and the appended claims, the word “distal” refers to direction towards a work site, and the word “proximal” refers to a direction away from the work site. Thus, for example, the end of a medical device that is closest to the target tissue would be the distal end of the medical device, and the end opposite the distal end would be the proximal end of the medical device.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various spatial positions and orientations. The combination of a body's position and orientation defines the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, medical instrument, and variants thereof, can be interchangeably used.

While certain exemplary examples of the present disclosure have been described and shown in the accompanying drawings, it is to be understood that such examples are merely illustrative of and not restrictive on the broad disclosure herein, and that the examples of the present disclosure should not be limited to the specific constructions and arrangements shown and described, since various other modifications may occur to those ordinarily skilled in the art.

1. An apparatus for movably coupling a robotic system to an airway management device, the apparatus comprising:
an overtube comprising a flexible tubular portion; and
a connection mechanism, comprising:
a first portion configured to receive the overtube;
a second portion configured to be connected with a proximal end of the airway management device;
a lumen defined within the first portion and second portion; and
a locking mechanism configured to connect to the flexible tubular portion of the overtube;
wherein when the overtube is connected with the connection mechanism, the flexible tubular portion of the overtube is within the lumen of the connection mechanism and extends distal to a distal end of the airway management device.

2. The apparatus of claim 1, further comprising a first seal, the first seal configured to seal a lumen of the flexible tubular portion of the overtube, wherein the first seal includes a cross-slit valve and a septum.

3. (canceled)

4. The apparatus of claim 2, further comprising a cap for the overtube, the cap comprising the first seal.

5. The apparatus of claim 1, further comprising a second seal, the second seal configured to seal a lumen of the connection mechanism, wherein the second seal includes a septum seal and a cross-slit valve.

6. (canceled)

7. The apparatus of claim 5, wherein the locking mechanism is configured to secure the second seal around the flexible tubular portion to prevent movement of the overtube.

8. The apparatus of claim 1, wherein the locking mechanism comprises:
a first lock part including a threaded base;
a second lock part including a threaded wall configured to engage the threaded base of the first lock part, the second lock part further including a foot extending inwardly from the threaded wall, the second lock part positioned adjacent a second seal; and
wherein relative movement between the threaded base of the first lock part and the threaded wall of the second lock part causes the foot of the second lock part to compress the second seal, thereby causing the second seal to expand radially inward to engage the flexible tubular portion of the overtube.

9. The apparatus of claim 8, wherein the locking mechanism further comprises an outer housing, and the second lock part comprises splines on an outside of the threaded wall, the splines configured to engage the outer housing so that the second lock part cannot rotate relative to the outer housing.

10. The apparatus of claim 1, wherein the connection mechanism is configured to be at least partially disconnected from the robotic system when motion between the robotic system and the airway management device includes an above-threshold displacement of the airway management device relative to the robotic system.

11.-13. (canceled)

14. A medical system comprising:
a robotic system configured to drive a flexible elongate device;
an airway management device, comprising:
a cylindrical passageway configured to couple with the robotic system; and
an engagement portion attached to the cylindrical passageway;
wherein the engagement portion is configured to engage a patient anatomy proximal to the trachea; and
an overtube comprising:
a flexible tubular portion configured to be inserted within the cylindrical passageway of the airway management device, the flexible tubular portion being configured to extend distal to the engagement portion of the airway management device when inserted within the cylindrical passageway of the airway management device, the flexible tubular portion structurally supporting a flexible elongate device when the flexible elongate device is received within flexible tubular portion.

15. The medical system of claim 14, wherein the robotic system includes an instrument carriage and a flexible elongate device guide configured to provide lateral support to the flexible elongate device during movement of the instrument carriage, the flexible elongate device being configured to accommodate the overtube.

16. (canceled)

17. The medical system of claim 14, further comprising
a connection mechanism, comprising:
a first portion configured to receive the overtube;
a second portion configured to be connected with a proximal end of the airway management device;
a lumen defined within the first portion and second portion; and
a locking mechanism configured to connect to the flexible tubular portion of the overtube, the locking mechanism being configured to secure a second seal around the flexible tubular portion to prevent movement of the overtube.

18. The medical system of claim 14, further comprising:
a first seal, the first seal configured to seal a lumen of the flexible tubular portion of the overtube; and
a second seal, the second seal configured to seal a lumen of a connection mechanism.

19.-23. (canceled)

24. The medical system of claim 17, the locking mechanism comprising:
a first lock part including a threaded base; and
a second lock part including a threaded wall configured to engage the threaded base of the first lock part, the second lock part further including a foot extending inwardly from the threaded wall, the second lock part positioned adjacent a second seal;
wherein relative movement between the threaded base of the first lock part and the threaded wall of the second lock part causes the foot of the second lock part to compress a second seal, thereby causing the second seal to expand radially inward to engage the flexible tubular portion of the overtube.

25. The medical system of claim 24, wherein the locking mechanism further comprises an outer housing, and the second lock part comprises splines on an outside of the threaded wall, the splines configured to engage the outer housing so that the second lock part cannot rotate relative to the outer housing.

26. The medical system of claim 17, wherein the connection mechanism is configured to be at least partially disconnected from the robotic system when motion between the robotic system and the airway management device includes an above-threshold displacement of the airway management device relative to the robotic system.

27.-29. (canceled)

30. A method of positioning a flexible elongate device, the method comprising:
providing a connection mechanism having a first portion and a second portion;
connecting the second portion of the connection mechanism to an airway management device;
inserting, using a robotic system, a distal portion of the flexible elongate device into an anatomy a first distance such that the flexible elongate device extends through the connection mechanism and extends distal to the airway management device;
extending an overtube over the flexible elongate device such that a flexible tubular portion of the overtube extends through the connection mechanism and extends distal to the airway management device; and
further inserting, using the robotic system, the flexible elongate device into the anatomy beyond the first distance, wherein the flexible tubular portion of the overtube provides structural support for the flexible elongate device distal to the airway management device.

31. The method of claim 30 further comprising locking the flexible tubular portion of the overtube to the connection mechanism including sealing, using a second seal, a lumen of the connection mechanism.

32. (canceled)

33. The method of claim 31, wherein the connection mechanism includes a locking mechanism, the locking mechanism including a first lock part including a threaded base, a second lock part including a threaded wall configured to engage the threaded base of the first lock part, and a foot extending inwardly from the threaded wall; and further comprising causing relative movement between the threaded base of the first lock part and the threaded wall of the second lock part to cause the foot of the second lock part to compress the second seal, thereby causing the second seal to expand radially inward to engage the flexible tubular portion of the overtube.

34. The method of claim 30, wherein the robotic system includes an instrument carriage and a flexible elongate device guide configured to provide lateral support to the flexible elongate device during movement of the instrument carriage; and further comprising:
extending the flexible elongate device guide when the flexible elongate device is inserted into the anatomy the first distance; and
retracting the flexible elongate device guide to enable insertion of the overtube over the flexible elongate device and re extending the flexible elongate device after insertion of the overtube.

35. (canceled)

36. The method of claim 30, wherein extending the overtube over the flexible elongate device does not result in extending the flexible tubular portion of the overtube distal to the flexible elongate device.
